(19) United States
(12) Patent Application Publication
(10) Pub. No.: US 2010/0030029 A1
(43) Pub. Date: Feb. 4, 2010

(54) Instrument for endoscopic surgery with knee in shaft and shaft stable handpiece

(76) Inventor: Harold A. Markham, Beverly Hills, CA (US)

Correspondence Address: Donald D. Mon, Suite 303, 750 East Green Street, Pasadena, CA, 91101, US

(21) Appl. No.: 12/221,592

(22) Filed: Aug. 4, 2008

(51) Int. Cl. A61B 1/01

(52) U.S. Cl. 600/146

(57) ABSTRACT

An endoscopic surgical instrument having a handpiece supporting an operating shaft. Said shaft having a knee enabling its distal end to bend away from the shaft axis, with its operative elements at the distal end. The handpiece has a stationary rest for the web of the thumb, and a four link engagement for the fingers, enabling actuation of the instrument without off-axis whipping motion.

### FIELD OF THE INVENTION

An endoscopic instrument with an elongated shaft for insertion into a body cavity. there to perform surgical procedures, such as cutting, grasping, occlusion, and clipping, for example. This shaft incorporates a knee which enables off-axis procedures, and a handpiece which when manipulated does not inherently cause the shaft to whip away from a pre-selected aimed axis, thereby freeing the surgeon from the attention otherwise needed to compensate for this undesirable event.

### BACKGROUND OF THE INVENTION

This invention relates to endoscopic instruments of the class which includes a shaft whose distal end is inserted into a body cavity. At its distal end the instrument can include any one of a wide variety of active elements. At its proximal end there is a handpiece for actuation of the instrument by the surgeon.

This general type of instrument is well-known and is in widespread usage. The events at the site where the operative elements accomplish their function are observed through a separate telescope system. Manipulation of the handpiece places the operative elements in an appropriate location and orientation and then is actuated to cause it to perform some surgical function. Such functions as cutting, grasping, occlusion and clipping are commonplace.

Conventional endoscopic procedures require a plurality of entry ports for various instruments. For example, one for inflation, another for the telescope, and yet another for the operative elements themselves. Each of the entry ports must be individually opened and closed, leaving scars which many patients find objectionable.

In response, a single port procedure has been developed in which the individual parts are all installed through a single entry port, preferably at the navel. These are inserted through a cannula which holds the port open and through which all instrumentation is passed.

This single port procedure with a conventional instrument limits the opportunities for off-axis procedures, because the operative elements are at the end of an inflexible, unbendable shaft. To perform a procedure that involves action off to the side of the cannula is not possible with conventional instruments. The instrument's field is limited to manipulation of the cannula, or of making the cannula passage much larger than the diameter of the shaft.

It is an object of this invention to provide in a single instrument the versatility to function in single port procedures as well as in procedures in which this instrument is the sole occupant of a cannula.

It is another object of this invention to provide the shaft with a knee along its length such that the distal end of the shaft with its operative elements can be pointed. toward and disposed at a location to the side of the axis of the shaft. The extent of the bend is selectable by means convenient to the surgeon's hand.

Another shortcoming of the prior art of hand-manipulated instruments is the conventional use of a scissor-like hand fatiguing arrangement in which the thumb is engaged to one arm of a handpiece, and a finger is engaged in the other arm, usually both with a loop.

One of the arms is fixed to the shaft, and the other is connected to a movable part of the shaft. The surgeon holds the shaft with its axis aligned with a place to be treated, and then the operative action is exerted. The problem is, that this actuation tends to whip the shaft away from its intended aimed direction. Then, in addition to everything else the surgeon has to do, he must manipulate the entire assembly to direct it correctly. This is a profound distraction at a critical time.

It is an optional object of this invention to provide an ergodynamic handpiece which, when the palm and the fingers are brought together, the body of the instrument supporting the shaft, and thereby the shaft itself will not be substantially tilted.

This handpiece enables the ready substitution of different single use shafts and operative elements, eliminating fear of secondary infection.

While this invention provides many advantages to the single port system, it is applicable to endoscopic instruments generally, including those which utilize multiple ports.

### BRIEF DESCRIPTION OF THE INVENTION

An endoscopic instrument according to this invention includes a handpiece for the surgeon to hold, and with it to manipulate the instrument. The handpiece includes an active and an inactive branch which branches are hinged together. The active branch of the handpiece is forward of the inactive branch. The inactive branch is supported by the web of the palm, and is fixed to the frame while the active branch is engaged by the fingers for actuation, and is movably mounted to the frame. This contrasts with conventional handpieces in which the thumb is used for actuation.

A shaft includes an active portion and an inactive portion. The inactive branch of the handpiece carries or is receptive to a mount. Preferably the mount receives and holds the inactive portion of the shaft. The active branch of the handpiece carries a socket that engages the active portion of the shaft. Accordingly, scissor-like movement of the handpiece will reciprocally shift the active portion of the shaft relative to its inactive portion. The inactive portion of the shaft remains stationary along with the inactive branch of the handpiece to which it is connected.

According to this invention, the active and inactive portions of the shaft are slidably coupled together. One of the shaft portions includes a longitudinal re-entrant slot, and the other includes an engaging re-entrant key.

The active element at the distal end of the shaft includes two operative parts. In cutting operations these operative parts might for example be scissor blades. In a gripping operation they might be jaws. These are operatively coupled to each other and pivoted to respective shaft portions such that reciprocation of the active portion of the shaft relative to the inactive portion results in opening and closing movement of the parts as directed by the surgeon.

The shaft is rotatably secured in the mount so that it can be rotated to suit the surgeon's requirements for positioning at the surgical site.

According to a feature of this invention, both shaft portions are split into two segments to form a knee. The segments of the active portion are coupled by a flexible elastic sleeve which enables the bending of the knee, and which tends to bias the segments toward the central axis of the shaft. The segments of the inactive portion are connected to each other by a hinge pin.

A reel that is rotatably attached to the frame carries a flexible inextensible filament. The filament is connected to the proximal and distal segments of the inactive portion, but is free from them for a distance across the knee joint. Pulling on the filament will cause the distal segments to be tilted away from the central axis of the instrument, thereby enabling procedures to be undertaken at the side of the instrument. The actuation caused by manipulation of the handpiece continues through the knee.

According to an optional feature of the invention the handpiece includes a frame to which the shaft is removably and rotatably attachable. The frame further includes a fixed palm rest. A four-link finger loop is mounted to the-frame.

The finger loop includes a fixed first link that is part of the frame. A second link is hinged to the frame at one end of the first link, The second link extends from one side of a first pivot to connect to the active portion of the shaft. It extends in a second direction and is pivoted to a second pivot. A third link forms part of the finger engagement for actuation of the active portion of the shaft. A fourth link extends from the third link to the first link, pivoted to both of them.

All of the links are rigid, and all are pivoted to their next-engaged links.

When this arrangement is manipulated, the finger loop moves substantially parallel to the shaft axis in opposition to the thumb or its web. A bias spring is used to force the actual portion of the shaft in a preferred direction. This reduces, nearly entirely, the tendency of the shaft to whip (tilt) when the fingers are moved toward or away from the thumb.

The above and other features of this invention will be fully understood from the following detailed description and the accompanying drawings, in which:

### BRIEF DESCRIPTION OF THE DRAWINGS

FIG. 1 is a side view of the presently preferred embodiment of the invention;

FIG. 2 is a portion of the back view of the handpiece in FIG. 1;

FIG. 3 is a cross-section taken at line 3-3 in FIG. 1;

FIG. 4 is a cross-section taken at line 4-4 in FIG. 1;

FIG. 5 is a side view of the knee in axial alignment.

FIG. 6 is a view as in FIG. 5, which the knee bent;

FIG. 7 shows the knee and one set of operative elements;

FIG. 8 is a cross-section of a mount; and

FIG. 9 is a top view of FIG. 8.

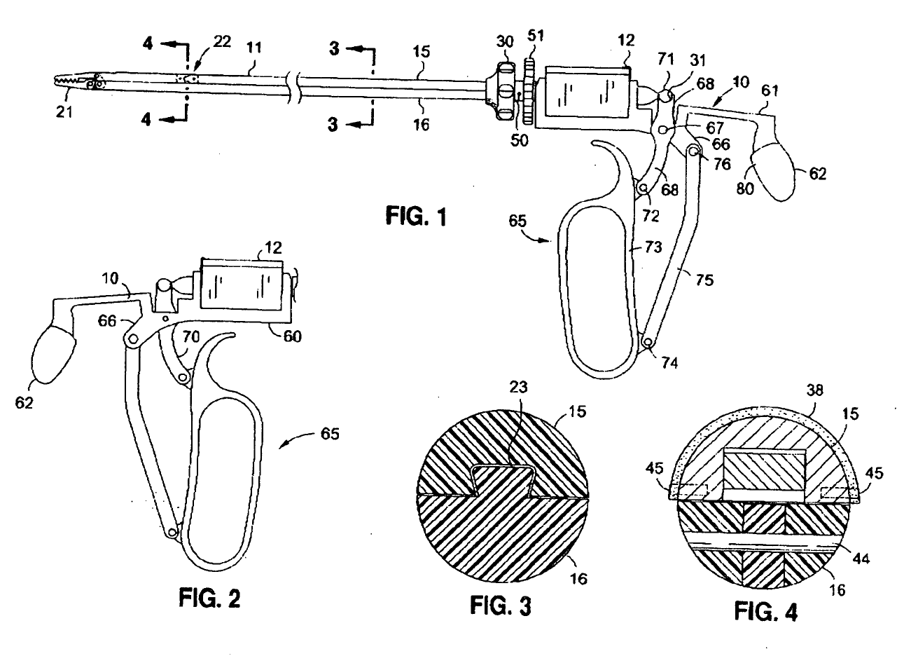

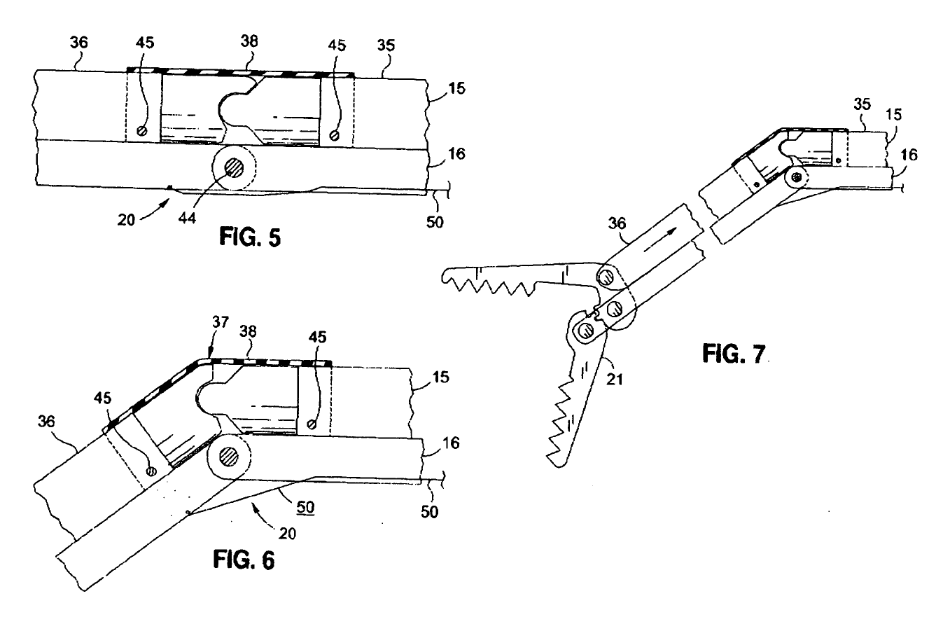

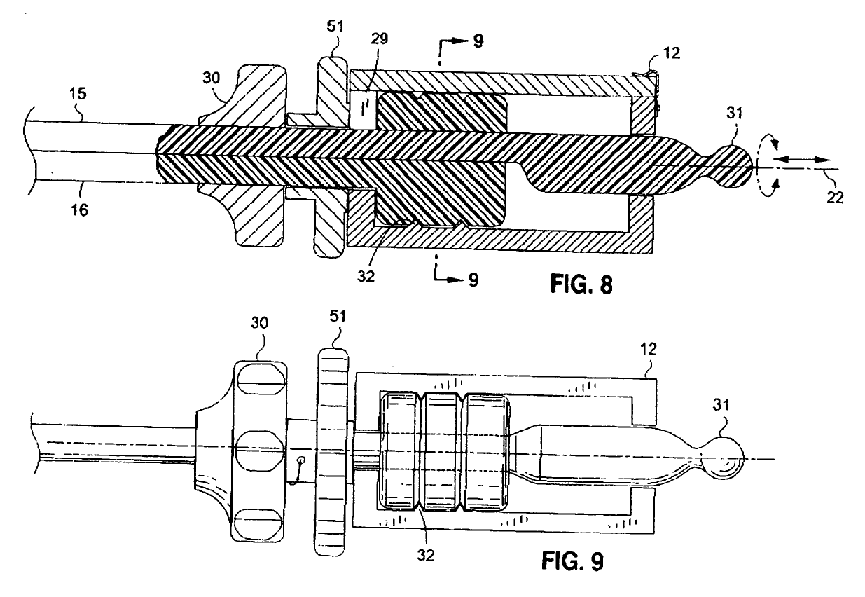

### DETAILED DESCRIPTION OF THE INVENTION

This instrument includes a handpiece 10 and a shaft 11. The handpiece includes a mount 12 to removably hold the shaft. The shaft includes an active portion 15 and an inactive portion 16 which are held in a close, sliding assembly.

The shaft is rotatable when in the mount. The shaft further includes a knee 20, and operative elements 21 at its distal end. These elements will now be described in detail.

Shaft 11 has a central axis 22 along which active portion 15 is axially slidably relative to inactive portion 16. These portions are held together by a tongue 23 on the inactive portion in a re-entrant groove in the active portion.

Mount 12 holds the shaft assembly to itself by means of a set of clips (not shown). The shaft assembly is rotatable in the mount and is removable and replaceable in it. They extend from a group of alignment bearings with inactive portion and pass through a slot 29 in one side of the mount. A wheel 30 is mounted to the assembly. It is connected to the inactive portion of the shaft and permits the separate axial movement of the active portion for instrument actuation while enabling it to rotate the assembly of the two portions to the operative site. Tongues 32 in the wall of the mount prevents axial movement of the inactive portion. They do not affect the active portion which is freely slidable. It is convenient to provide some kind of limited restraint on the rotation of the wheel so that a selected angle will be maintained until a strong enough torque is exerted on it to overcome the restraint.

The active portion of the shaft carries an ▪olive▪ 31 for engagement with the handpiece, as will later be described.

Active portion 15 includes a proximal segment 35 and a distal segment 36. These segments adjoin one another at a joint 37. They are joined together by a patch 38 of flexible resilient material such as silicone rubber. This patch-is held to the active portion by a pin 45 on each side of the active portion and at each end of the adjacent segments. The patch is the only physical connection between the two segments of the active portion.

The segments of inactive portion 16 are joined together by a hinge pin 44. The patch on the active portion and the hinge on the inactive portion form the knee 20. The hinge pin limits the knee action to a plane normal to it, so the distal portions can be tilted away from the central axis. The elastic stretch in the patch tends to resist this motion and to restore the segments to their axially-aligned condition.

The tilt of the distal segment is caused by a filament 50 wound on a wheel 51 located adjacent to wheel 30. The filament extends through a passage in the inactive portion to a location proximally spaced from the hinge pin. It exits the passage and is externally connected to the distal segment of the inactive portion on the other side of the hinge pin. Thus, as shown in FIG. 6, wheel 51 can be turned to pull on the filament and cause both distal segments to tilt away from the axis. The movement of the distal segment of the distal portion is carried across the knee by the patch. A portion 58 of the filament will be outwardly spaced from the shaft when the knee is tilted, but this will not interfere with the alignment or use of the instrument, because this event will usually occur after the shaft has been rotated near to its desired adjustment.

Operative elements 21 may be of any type which can be mounted between an active and an inactive shaft segment. The means for mounting these elements are not unique to this invention. For purpose of example, reference may be made to applicant's co-pending U.S. patent application Ser. No. 11/895,160 filed Aug. 23, 2007 for full details which are incorporated herein by reference in their entirety.

For purposes of this invention, it is sufficient to observe that when the active portion is shifted relative to the inactive portion, the operative element will respond, whether the segments are aligned or not. The segment of the active portion will still move with one another Bending the knee will cause slight movement of the operative element, but this is readily compensated by manipulation of the handpiece.

Mount 12 is a removable part of the handpiece, in turn forming part of a rigid frame 60. A distal extension 61 of the frame includes a palm rest 62, which can be engaged by the web of the rest, or can be modified to form a grip for the thumb. Either way, the thumb (or web) can be pressed against the frame for actuation purposes.

A finger engagement 65 is formed as part of a four-link system. First link 66 is a rigid part of the frame. A first pivot 67 on the frame at one end of the first link pivotally mounts a second link 68.

Second link 68 has two segments: one operative arm 69 on one side of the first pivot, and a rigid part 70 on the other. Arm 69 has a cup 71 receptive of the olive at the end of the active portion.

The second link extends to a second pivot 72, which pivot mounts a third link 73. This link 73 extends to a third pivot 74 to which is mounted a fourth link 75.

The fourth link is pivotally connected to the first link by a fourth pivot 76 on the first link.

These links provide a controlled movement of the third link, which moves in a nearly straight path parallel to the axis of the shaft.

The third link includes a palm rest 80. It will be seen that with the palm on the web, and the fingers on the third link, the third link can be brought toward and away from the thumb without a whipping movement of the shaft While there may be some tilting action of the shaft, it is minor and can readily be compensated for.

A flat return spring (not shown) can be provided between the segments of the shaft to return the segments to their axial position if the patch is not sufficient for the purposes.

Each of the two improvements to the shaft and to the handpiece can be used without the other, but when combined in an instrument there results a conveniently adjusted instrument with off-axis capabilities and without substantial whip action when the instrument is actuated.

Should electro surgery be desired, appropriate connectors to the frame, and circuitry through the shaft may readily be provided as in the-previously mentioned patent application Ser. No. 11/895,160.

This invention is not to be limited by the embodiment shown in the drawings and described in the description, which is given by way of example and not of limitation, but only in accordance with the scope of the appended-claims.

I claim:

1. In an endoscopic instrument comprising a handpiece, a rigid shaft having an axis, said shaft including an active and an inactive portion joined together for relative axial movement, and an operative element at the distal end of said shaft, the improvement comprising:
each of said portions divided into a distal segment and a proximal segment, said segments of the inactive portion being joined by a hinge pin, and said segments of said active portion being joined by a flexible elastic patch whereby to form a knee which bends at said hinge and a filament retained in and exists from a channel in said inactive portion externally connected to said distal segment, thereby extending free across the knee; said filament being attached to a reel rotatably mounted to said handpiece adapted to pull on the filament to bend the knee, and a handpiece rotatably mounting said shaft, said handpiece including a frame, an actuating arm adapted for connection to the active portion of the shaft, said frame including a palm rest at its proximal end; and a four link system for actuation by the fingers, said four link system comprising, a first rigid link on said frame, a first pivot at one end of said first link, a second rigid link pivoted to said first pivot with said arm from one side of said first pivot, said second link extending to a second pivot, a third link pivoted and said second pivot and a third pivot and a fourth link pivoted to said third pivot on to a fourth pivot on said first link, said third link being provided for engagement by fingers while the palm engages the palm rest.

2. An endoscopic instrument according to claim 1 in which said active and inactive portions are joined by an axially-extended tongue and reentrant groove, said shaft being adjustably rotatable in said instrument.

3. An endoscopic instrument according to claim 1 in which said operative element is adapted to at least one of the following functions, cutting, grasping, occlusion and clipping.

4. An endoscopic instrument according to claim 1 in which said shaft is held by a mount, which in turn is removably held to the handpiece for ready substitution.

5. In an endoscopic instrument having a handpiece, a rigid shaft having an axis, said shaft including an active portion and an inactive portion joined together for relative axial movement, and an operative element at the distal end of said shaft, the improvement comprising:
dividing each of said portions into a distal segment and a proximal segment, joining said segments of the inactive portion with a hinge pin, and said segments of said active portion with a flexible elastic patch whereby to form a knee which bends at said hinge and finally to restore the portion to an aligned condition; and
a filament retained-in a channel in said inactive portion externally connected to said distal segment; said filament being attached to a reel rotatably mounted to said handpiece adapted to pull on the filament to bend the knee, said filament freely extending across the knee.

6. A handpiece rotatably to mount a shaft, said shaft having an active portion and an inactive portion joined together for relative sliding axial movement;
said handpiece including a frame, and an actuating arm adapted for connection to the active portion, said frame including a palm rest at its proximal end; and
a four link system for actuation by the fingers, said four link system comprising:
a first rigid link on said frame, a first pivot at one end of said first link;
a second rigid link pivoted to said first pivot with said arm from one side of said first pivot, said second link extending to a second pivot;
a third link pivoted to said second pivot and a third pivot and;
a fourth link pivoted to said third pivot and to a fourth pivot on said first link;
said third link being provided for engagement by fingers while the palm engages the palm rest.
